(19) United States
(12) Patent Application Publication
(10) Pub. No.: US 2023/0301577 A1
(43) Pub. Date: Sep. 28, 2023

(54) MONITORING SYSTEM, ATRIAL FIBRILLATION COMPREHENSIVE MANAGEMENT METHOD AND MONITORING DATA DISPLAY METHOD

(71) Applicant: SHENZHEN MINDRAY BIO-MEDICAL ELECTRONICS CO., LTD., Shenzhen (CN)

(72) Inventors: Mengxing LIU, Shenzhen (CN); Zehui SUN, Shenzhen (CN); Zehong GUAN, Shenzhen (CN); Xianliang HE, Shenzhen (CN); Wenyu YE, Shenzhen (CN)

(73) Assignee: SHENZHEN MINDRAY BIO-MEDICAL ELECTRONICS CO., LTD., Shenzhen (CN)

(21) Appl. No.: 18/203,079

(22) Filed: May 30, 2023

Related U.S. Application Data
(63) Continuation of application No. PCT/CN2020/132963, filed on Nov. 30, 2020.

(51) Int. Cl. A61B 5/361; A61B 5/339

(52) U.S. Cl. CPC A61B 5/361; A61B 5/339

(57) ABSTRACT

A monitoring system, an atrial fibrillation comprehensive management method and a monitoring data display method are disclosed. The atrial fibrillation comprehensive management method includes acquiring monitoring data of at least two vital sign parameters of a patient, where the at least two vital sign parameters comprise an electrocardiogram parameter; acquiring information of an atrial fibrillation event occurring in the patient; and displaying an atrial fibrillation comprehensive view according to the acquired information of the atrial fibrillation event and the acquired monitoring data of the at least two vital sign parameters. The atrial fibrillation comprehensive view includes a trend graph of at least two vital sign parameters, an atrial fibrillation event trend graph and an atrial fibrillation load graph. Doctor can know a comprehensive situation of the patient from the atrial fibrillation comprehensive view and rapidly take a countermeasure, thereby improving the efficiency of atrial fibrillation management.

### CROSS-REFERENCE TO RELATED APPLICATIONS

This application is a continuation of International Application No. PCT/CN2020/132963, filed on Nov. 30, 2020. The entire contents of the above-referenced application are expressly incorporated herein by reference.

### TECHNICAL FIELD

The disclosure relates to the technical field of medical device, and more particularly to a monitoring system, atrial fibrillation comprehensive management method and monitoring data display method.

### BACKGROUND

AF, short for Atrial fibrillation, is one of the most common arrhythmias in cardiology. The incidence of atrial fibrillation gradually increases with age, reaching 7.5% in people over 80 years old. Atrial fibrillation is extremely common in cardiac intensive care, for example, the incidence of atrial fibrillation in patients with severe heart failure is as high as 55%. To detect and evaluate atrial fibrillation in a timely manner, medical staff often use bedside monitors to continuously monitor patients in real-time. The existing monitoring system usually only detects and warns the atrial fibrillation. For example, when the monitor detects the atrial fibrillation on the electrocardiogram through backend algorithms, it automatically pops up a string of “atrial fibrillation” in the alarm area and reminds medical staff to pay attention through sound and light indications. When atrial fibrillation stops and sinus rhythm resumes, the string of “atrial fibrillation stop” automatically pops up in the alarm area. In addition to the real-time alarms mentioned above, medical staff can also browse historical alarm data through the event list in the review interface. However, medical staff cannot make a comprehensive determination of patient solely based on atrial fibrillation alarms and electrocardiogram waveforms at the occurrence time of atrial fibrillation, so the efficiency of atrial fibrillation management by medical staff is relatively low.

This disclosure mainly provides a monitoring system, an atrial fibrillation comprehensive management method, and a monitoring data display method to improve the efficiency of atrial fibrillation management.

### SUMMARY

An embodiment provides an atrial fibrillation comprehensive management method, including:
acquiring monitoring data of at least two vital sign parameters of a patient, wherein the at least two vital sign parameters include electrocardiogram parameter(s);
acquiring information of an atrial fibrillation event which occurs in the patient;
displaying an atrial fibrillation comprehensive view, according to the information of the atrial fibrillation event and the monitoring data of the at least two vital sign parameters; wherein the atrial fibrillation comprehensive view includes trend graphs of the at least two vital sign parameters, a trend graph of the atrial fibrillation event, and an atrial fibrillation burden graph;
wherein the trend graphs of the at least two vital sign parameters are configured to present changes of the at least two vital sign parameters during a preset atrial fibrillation monitoring period, the trend graph of the atrial fibrillation event is configured to present the atrial fibrillation event that occurs in the patient during the preset atrial fibrillation monitoring period, and the atrial fibrillation burden graph is configured to present atrial fibrillation burden within at least one time period of minimum unit, during the preset atrial fibrillation monitoring period;
wherein the method further includes:
simultaneously displaying the trend graphs of the at least two vital sign parameters, the trend graph of the atrial fibrillation event, and the atrial fibrillation burden graph, so as to jointly present a correlation between at least two of: at least one vital sign parameter, the atrial fibrillation event that occurs in the patient, and the atrial fibrillation burden, at any one or more time points or within any one or more time periods during the preset atrial fibrillation monitoring period.

An embodiment provides a monitoring data display method, including:
acquiring monitoring data of at least one vital sign parameter of a patient, wherein the at least one vital sign parameter includes electrocardiogram parameter(s);
acquiring information of an atrial fibrillation event which occurs in the patient;
displaying an atrial fibrillation comprehensive analysis interface according to the information of the atrial fibrillation event and the monitoring data of the at least one vital sign parameter, wherein the atrial fibrillation comprehensive analysis interface includes a parameter trend area and/or an information statistics area,
wherein the parameter trend area displays a trend graph of the at least one vital sign parameter, and the parameter trend area further displays at least one of: a trend graph of the atrial fibrillation event and an atrial fibrillation burden graph; wherein the trend graph of the at least one vital sign parameter is configured to present change(s) of the at least one vital sign parameter during a preset atrial fibrillation monitoring period, the trend graph of the atrial fibrillation event is configured to present the atrial fibrillation event that occurs in the patient during the preset atrial fibrillation monitoring period, and the atrial fibrillation burden graph is configured to present atrial fibrillation burden during at least one time period of minimum unit within the preset atrial fibrillation monitoring period;
wherein the method further includes:
displaying the trend graph of the at least one vital sign parameter simultaneously with at least one of the trend graph of the atrial fibrillation event and the atrial fibrillation burden graph, so as to jointly present a correlation between the at least one vital sign parameter, and at least one of the atrial fibrillation event that occurs in the patient and the atrial fibrillation burden, at any one or more time points or within any one or more time periods during the preset atrial fibrillation monitoring period;
wherein the information statistics area displays at least one of followings: statistical information for the atrial fibrillation, statistical information for the at least one vital sign parameter, and statistical information for typical event(s).

An embodiment provides an atrial fibrillation comprehensive management method, including:
acquiring monitoring data of at least one vital sign parameter of a patient, wherein the at least one vital sign parameter includes electrocardiogram parameter(s);
acquiring information of an atrial fibrillation event which occurs in the patient;
outputting an electronic version or a paper version of an overview report according to the information of the atrial fibrillation event and the monitoring data of the at least one vital sign parameter, wherein the overview report includes a trend graph of the at least one vital sign parameter, as well as a trend graph of the atrial fibrillation event and/or an atrial fibrillation burden graph;
wherein the trend graph of the at least one vital sign parameter is configured to present change(s) of the at least one vital sign parameter during a preset atrial fibrillation monitoring period, the trend graph of the atrial fibrillation event is configured to present the atrial fibrillation event that occurs in the patient during the preset atrial fibrillation monitoring period, and the atrial fibrillation burden graph is configured to present atrial fibrillation burden during at least one time period of minimum unit within the preset atrial fibrillation monitoring period.

An embodiment provides a monitoring system, including:
a signal acquisition device, which is configured to acquire monitoring data of at least two vital sign parameters of a patient, wherein the at least two vital sign parameters include electrocardiogram parameter(s);
a processor, which is configured to detect atrial fibrillation according to the monitoring data, so as to acquire information of an atrial fibrillation event which occurs in the patient, or configured to acquire information of an atrial fibrillation event which occurs in the patient through the signal acquisition device; and configured to display, on a display interface of a display device, an atrial fibrillation comprehensive view, according to the information of the atrial fibrillation event and the monitoring data of the at least two vital sign parameters; wherein the atrial fibrillation comprehensive view includes trend graphs of the at least two vital sign parameters, a trend graph of the atrial fibrillation event, and an atrial fibrillation burden graph;
wherein the trend graphs of the at least two vital sign parameters are configured to present changes of the at least two vital sign parameters during a preset atrial fibrillation monitoring period, the trend graph of the atrial fibrillation event is configured to present the atrial fibrillation event that occurs in the patient during the preset atrial fibrillation monitoring period, and the atrial fibrillation burden graph is configured to present atrial fibrillation burden within at least one time period of minimum unit, during the preset atrial fibrillation monitoring period;
wherein the processor is further configured to:
simultaneously display the trend graphs of the at least two vital sign parameters, the trend graph of the atrial fibrillation event, and the atrial fibrillation burden graph, so as to jointly present a correlation between at least two of: at least one vital sign parameter, the atrial fibrillation event that occurs in the patient, and the atrial fibrillation burden, at any one or more time points or within any one or more time periods during the preset atrial fibrillation monitoring period.

An embodiment provides a monitoring system, including:
a signal acquisition device, which is configured to acquire monitoring data of at least one vital sign parameter of a patient, wherein the at least one vital sign parameter includes electrocardiogram parameter(s);
a processor, which is configured to detect atrial fibrillation according to the monitoring data, so as to acquire information of an atrial fibrillation event which occurs in the patient, or configured to acquire information of an atrial fibrillation event which occurs in the patient through the signal acquisition device; and configured to output an electronic version or a paper version of an overview report according to the information of the atrial fibrillation event and the monitoring data of the at least one vital sign parameter, wherein the overview report includes a trend graph of the at least one vital sign parameter, as well as a trend graph of the atrial fibrillation event and/or an atrial fibrillation burden graph;
wherein the trend graph of the at least one vital sign parameter is configured to present change(s) of the at least one vital sign parameter during a preset atrial fibrillation monitoring period, the trend graph of the atrial fibrillation event is configured to present the atrial fibrillation event that occurs in the patient during the preset atrial fibrillation monitoring period, and the atrial fibrillation burden graph is configured to present atrial fibrillation burden during at least one time period of minimum unit within the preset atrial fibrillation monitoring period.

An embodiment provides a computer-readable storage medium, including a program that can be executed by a processor to implement the method described above.

The monitoring system, atrial fibrillation comprehensive management method and monitoring data display method of the above embodiments, acquire monitoring data of at least two vital sign parameters of a patient, acquire information of an atrial fibrillation event which occurs in the patient; display an atrial fibrillation comprehensive view according to the information of the atrial fibrillation event and the monitoring data of the at least two vital sign parameters; wherein the atrial fibrillation comprehensive view includes trend graphs of the at least two vital sign parameters, a trend graph of the atrial fibrillation event, and an atrial fibrillation burden graph. In this way, doctors can understand the comprehensive situation of patient through the atrial fibrillation comprehensive view, which facilitates rapid response measures and improves the efficiency of atrial fibrillation management.

### BRIEF DESCRIPTION OF DRAWINGS

FIG. 1 is a structural block diagram of an embodiment of a monitoring system provided by this disclosure.

FIG. 2 is a schematic diagram of an embodiment of an atrial fibrillation comprehensive view in a monitoring system provided by this disclosure.

FIG. 3 is a partial enlarged view of various trend graphs in FIG. 2.

FIG. 4 is a structural block diagram of an embodiment of a processor in a monitoring system provided by this disclosure.

FIG. 5 is a flowchart of an embodiment of an atrial fibrillation comprehensive management method in a monitoring system provided by this disclosure.

FIG. 6 shows a parameter configuration interface displayed by an atrial fibrillation configuration unit through a display device in a monitoring system provided by this disclosure.

FIG. 7 shows a monitoring interface for multiple patients in a monitoring system provided by this disclosure.

FIG. 8 is a schematic diagram of an embodiment of a patient monitoring interface in a monitoring system provided by this disclosure.

FIG. 9 is a schematic diagram of an embodiment of a patient monitoring interface in a monitoring system provided by this disclosure.

FIG. 10 is a schematic diagram of an embodiment of an atrial fibrillation overview and an atrial fibrillation comprehensive view in a monitoring system provided by this disclosure.

FIG. 11 is a schematic diagram of an embodiment of an atrial fibrillation view segmented for time in a monitoring system of this disclosure.

FIG. 12 is a schematic diagram of an embodiment of an atrial fibrillation time-segmented view in a monitoring system of this disclosure.

FIG. 13 is a flowchart of an embodiment of a monitoring data display method in a monitoring system of this disclosure.

### DETAILED DESCRIPTION

This disclosure is further described in detail below through specific embodiments in combination with the accompanying drawings. In different embodiments, similar elements adopt associated similar reference numbers. In the following embodiments, many details are described in order to make this disclosure better understood. However, those skilled in the art can easily recognize that some of the features can be omitted in different cases, or can be replaced by other elements, materials and methods. In some cases, some operations related to this disclosure are not shown or described in the description, in order to avoid the core part of this disclosure being inundated by too many descriptions. For those skilled in the art, it is not necessary to describe these related operations in detail, as they can fully understand the relevant operations according to the description in this disclosure and the general technical knowledge in the art.

In addition, the characteristics, operations, or features described in the specification may be combined in any appropriate manner to form various embodiments. At the same time, the steps or actions in the method description can also be exchanged or adjusted in order in a manner obvious to those skilled in the art. Therefore, the various sequences in the description and the drawings are only for the purpose of clearly describing a certain embodiment, and do not mean that they are necessary sequences. Unless it is otherwise specified that one of the sequences must be followed.

The terms “first”. “second”, etc., in the specification are operable to distinguish different objects, rather than to describe a specific order. In addition, the terms “connection”, and “linkage” mentioned in this disclosure include direct and indirect connection (linkage), unless otherwise specified.

The monitoring system, the atrial fibrillation comprehensive management method and the monitoring data display method provided by this disclosure, present information for atrial fibrillation event, vital sign parameter, atrial fibrillation burden and the likes, in a centralized manner through the design of the AF View, so as to help the medical staff to systematically evaluate the physical state of patient.

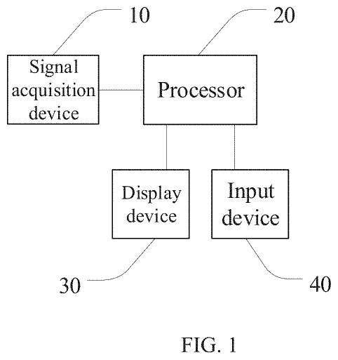

As shown in FIG. 1, the monitoring system provided by this disclosure includes a signal acquisition device 10, a processor 20, a display device 30, and an input device 40.

The input device 40 is configured to receive input from a user (usually an operator). For example, one or more of a mouse, a keyboard, a touch display, a trackball, a joystick, or the likes may be configured to receive instructions inputted by a user, or the likes. Users can perform input operation through the input device 40.

The display device 30 is configured to output information, such as visual information. Display device 30 can be a display device that only has display functions, or can be a touch-control display device. It can be seen that the display device 30 and the input device 40 are human-machine interaction devices of the monitoring system. The human-machine interaction device can not only receive user inputted instructions, but also display visual information.

The signal acquisition device 10 is configured to acquire monitoring data, such as monitoring data of at least two vital sign parameters of a patient, wherein the at least two vital sign parameters include electrocardiogram parameter(s) (electrocardiogram signals).

The monitoring system can be any of: a monitor, a local central station, a remote central station, a cloud service system, or a mobile terminal, and the corresponding signal acquisition device 10 acquires the monitoring data in different ways. For example, if the monitoring system is a monitor, the signal acquisition device 10 can use sensors to monitor the vital sign parameters of the patient to obtain monitoring data of the vital sign parameter. The monitoring data of the vital sign parameter can include at least one of: types of vital sign parameters (such as at least one type of: ECG, respiration, non-invasive blood pressure, oxygen saturation, pulse, body temperature, invasive blood pressure, carbon dioxide at end respiratory, respiratory mechanics, anesthetic gas, cardiac output, Bispectral index of EEG, etc.), values of vital sign parameters, and trends of changes of values of vital sign parameters. For example, the monitoring system can be a local central station, a remote central station, a cloud service system, or a mobile terminal, then the signal acquisition device 10 can be a communication device or interface configured to communicate with the monitor to obtain the aforementioned monitoring data from the monitor.

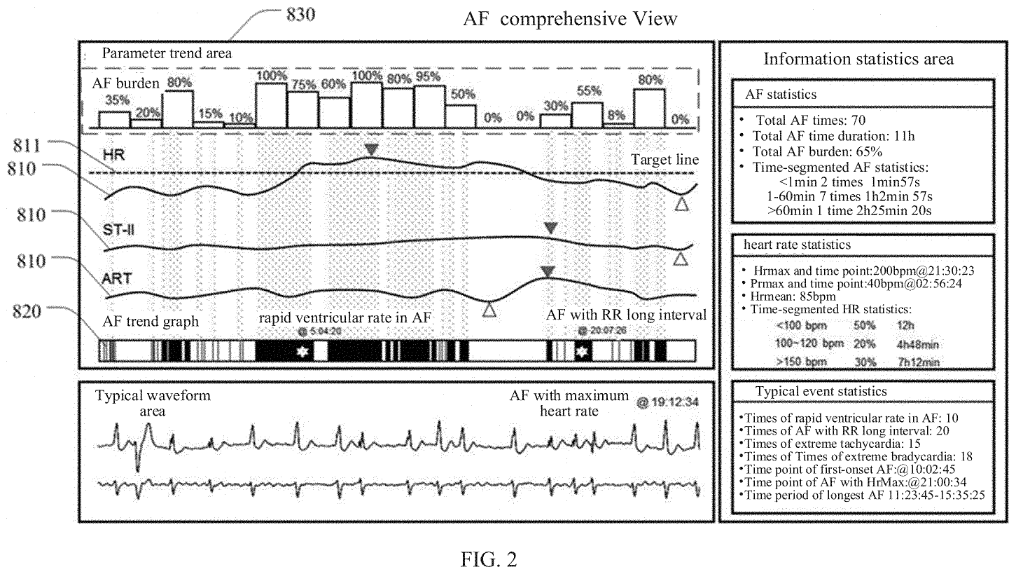

The processor 20 can be constructed to detect atrial fibrillation according to the monitoring data acquired by the signal acquisition device 10 and obtain an atrial fibrillation analysis result, which at least includes information of an atrial fibrillation event which occurs in the patient. Of course, since the existing monitor can record the atrial fibrillation event, the processor 20 can also acquire information of the atrial fibrillation event which occurs in the patient, from the monitor through the signal acquisition device 10. The processor 20 generates an atrial fibrillation comprehensive view according to the information of the atrial fibrillation event and the monitoring data of at least two vital sign parameters, and outputs the atrial fibrillation comprehensive view. The processor 20 outputs the atrial fibrillation comprehensive view, such as through displaying the atrial fibrillation comprehensive view, outputting an electronic overview report, and printing a paper overview report through a connected printing device. In this embodiment, displaying the atrial fibrillation comprehensive view is used as an example for explanation, as shown in FIG. 2. The atrial fibrillation comprehensive view includes trend graphs 810 of at least two vital sign parameters, a trend graph 820 of the atrial fibrillation event, and an atrial fibrillation burden graph 830. It should be understood that in addition to information related to the atrial fibrillation event (such as the atrial fibrillation comprehensive view), the overview report can also include information other than about the atrial fibrillation event, such as information characterizing other vital signs, diagnostic content, test results, etc., of the patient.

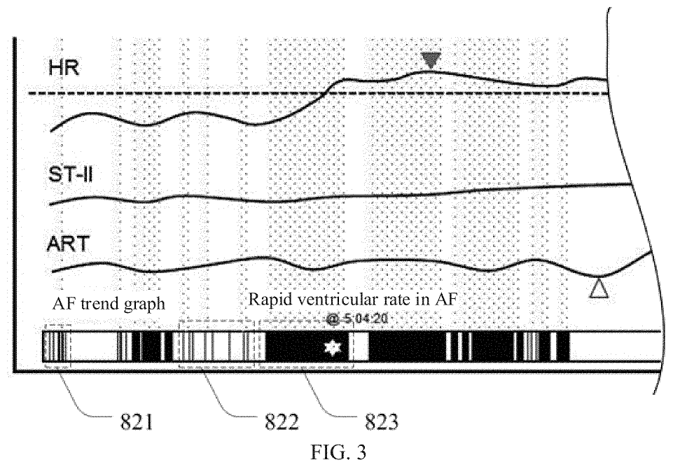

Trend graph is also known as transition diagram, run graph, chain diagram, trend graph, and so on. The trend graph can be configured to reflect the relationship between one or more variables and time, that is, the trend of the development of one or more variables over time. For example, a trend graph can use time as the horizontal axis and the observed variable as the vertical axis to observe the trends and/or biases of changes and developments of the variable. The time on the horizontal axis can be seconds, minutes, hours, days, months, years, etc., and each time point should be continuous and uninterrupted. The observation variable on the vertical axis can be absolute quantity/absolute value, average value, incidence, etc. In this disclosure, the trend graphs of the vital sign parameters can be configured to reflect the trend of a certain vital sign parameter over time. For example, the parameter value of the vital sign parameter continuously changes over time. The parameter value can be either absolute value which are acquired at a certain sampling rate or average values which are acquired at a certain sampling rate and calculated at each fixed time period. Therefore, for vital sign parameters, the “variable” in the trend graph is usually the parameter value of the vital sign parameter. The trend graph of the atrial fibrillation event can be configured to reflect the trend of the atrial fibrillation event over time, such as the occurrence pattern, development trend, incidence, trend, and so on. Here, “incidence” can be an occurrence frequency of event within a time period of minimum unit, while the occurrence pattern, development trend, trend, etc., are not limited to the occurrence frequency, but can be marked according to the actual situation of the event. For example, for each atrial fibrillation event, special graphics/symbols are configured to mark it. When the atrial fibrillation event is a continuous and long-term atrial fibrillation event, the occurrence time duration of the atrial fibrillation event can be reflected by a spanning length of the horizontal axis (i.e., timeline) using graphics/symbols. In the following, the occurrence patterns, development trends, incidences, trends, and so on of atrial fibrillation events reflected in various trend graphs are described in conjunction with FIG. 3.

The trend graphs 810 of the at least two vital sign parameters are configured to show the changes of at least two vital sign parameters during the preset atrial fibrillation monitoring period. That is, the trend graph 810 of vital sign parameter is configured to display the changes of this vital sign parameter during the preset atrial fibrillation monitoring period. The trend graph of vital sign parameter displayed in the atrial fibrillation comprehensive view can include at least one of: a trend graph of heart rate, a trend graph of pulse rate, a trend graph of blood oxygen, a trend graph of non-invasive blood pressure, a trend graph of invasive blood pressure, a trend graph of respiration, a trend graph of body temperature, a trend graph of cardiac output per stroke, a trend graph of cardiac output, a trend graph of ST segment of electrocardiogram, a trend graph of QT interval of electrocardiogram, a trend graph of blood sugar, a trend graph of cerebral oxygen and a trend graph of urine volume. The trend graph of vital sign parameter can reflect the trend of change of vital sign parameter. It can be one of a curve graph, a histogram, a bar graph, a box graph, a scatter graph, and a broken line graph, or various combinations of the curve graph, histogram, bar graph, box graph, scatter graph, and broken line graph. In this embodiment, the trend graph 810 of vital sign parameter is illustrated using a curve graph as an example.

The trend graph 820 of the atrial fibrillation event is configured to present the atrial fibrillation event that occurs in the patient during the preset atrial fibrillation monitoring period, for example, to present changes in occurrence count and/or frequency of atrial fibrillation events during the preset atrial fibrillation monitoring period. The trend graph 820 of the atrial fibrillation event can reflect the trend of change of the atrial fibrillation event. It can be one of curve graph, histogram, bar graph, box graph, scatter graph, line graph, or various combinations of curve graph, histogram, bar graph, box graph, scatter graph, and a broken line graph. Through the trend graph 820 of the atrial fibrillation event, doctors can see whether the atrial fibrillation occurs during the time period they want to pay attention to, the distribution (frequency and time duration) of the atrial fibrillation event, and the occurrence or non-occurrence of atrial fibrillation, which is very convenient.

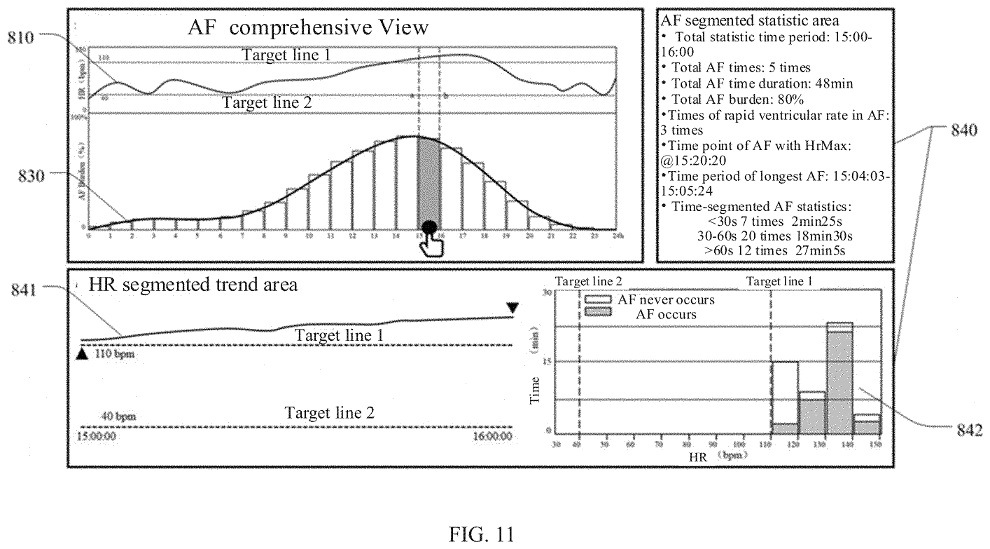

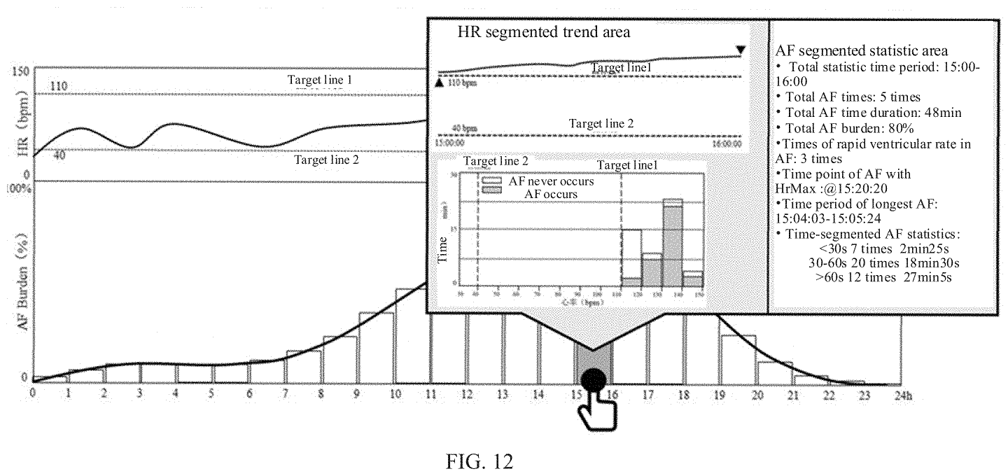

The atrial fibrillation burden graph 830 is configured to present the atrial fibrillation burden for at least one time period of minimum unit during the preset atrial fibrillation monitoring period. By using the atrial fibrillation burden graph 830, the changes of burden between each time period of minimum unit can be directly quantified. Doctors can see an accumulation degree of atrial fibrillation and specific amount of the burden, which is very convenient. The time period of minimum unit can be automatically preset by the system, or can be inputted or modified by the user. In the embodiment shown in FIG. 2, the time period of minimum unit is 1 hour. The time period of minimum unit can be the same as or different form an update cycle. This embodiment takes the previous as an example for explanation. FIG. 2 lists the atrial fibrillation burden graph 830, which is a statistical histogram, in which height of each bar in the histogram represents the atrial fibrillation burden within each time period of minimum unit. The maximum value of 100% means that the patient has been continuously experiencing atrial fibrillation within this time period of minimum unit, and width of the bar is the time duration for the time period of minimum unit, which can be set by the user. As shown in FIG. 11, a continuous curve is fitted along an upper edge of the histogram, representing the distribution of changes in the atrial fibrillation burden throughout the monitoring period.

Moreover, the trend graphs 810 of the at least two vital sign parameters, as well as the trend graph 820 of the atrial fibrillation event, and the atrial fibrillation burden graphs 830, are displayed simultaneously to facilitate the joint presentation of a correlation between at least two of: at least one vital sign parameter, the atrial fibrillation event occurs in the patient, and the atrial fibrillation burden, at any one or more time points or within any one or more time periods during the preset atrial fibrillation monitoring period.

Traditional monitoring devices are mainly limited to event alarms and reviews for atrial fibrillation management, and the reviewed content is mostly limited to simply presentation of the time point of alarm in the review list or provision of typical parameter (usually electrocardiogram) waveform(s) for user to view. This disclosure provides a more comprehensive method for managing atrial fibrillation according to electrocardiogram parameters, involving the joint presentation of vital sign parameter, atrial fibrillation event, and atrial fibrillation burden, and the interconnection and organic combination of various kinds of information. It can be seen that doctors can understand the comprehensive situation of patient through the atrial fibrillation comprehensive view, which facilitates rapid response measures and improves the efficiency of atrial fibrillation management.

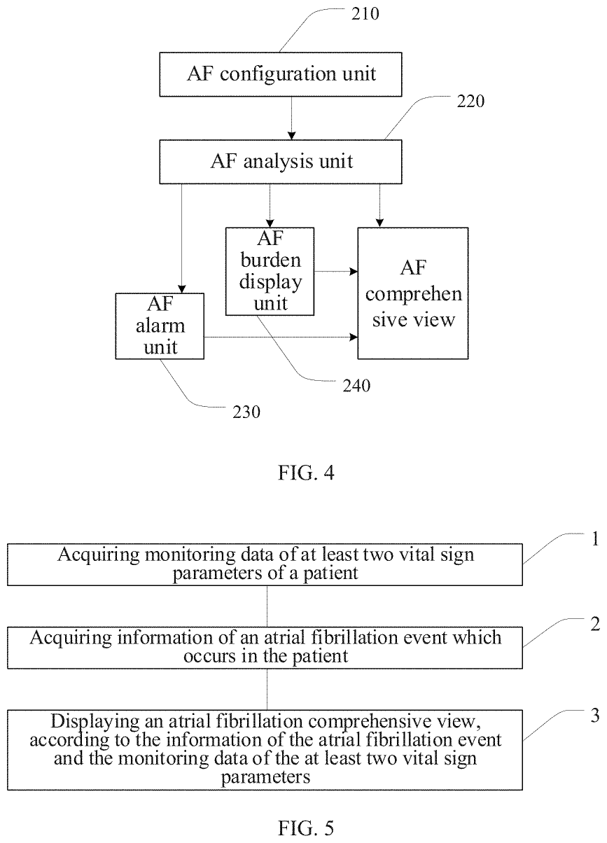

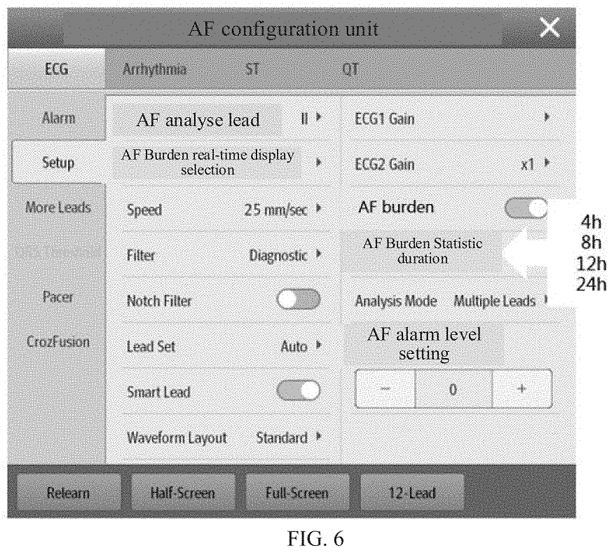

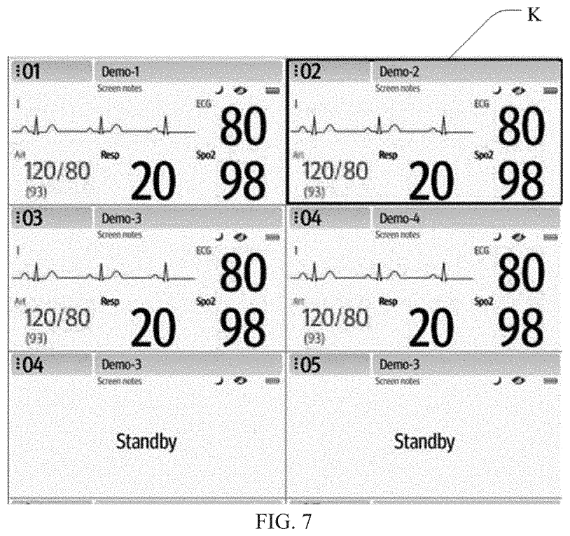

As shown in FIG. 4, the processor 20 includes an atrial fibrillation configuration unit 210, an atrial fibrillation analysis unit 220, an atrial fibrillation alarm unit 230, and atrial fibrillation burden display unit 240. The atrial fibrillation configuration unit 210 is configured to provide personalized configuration for atrial fibrillation analysis and management to medical staff, as shown in FIG. 6, including but not limited to lead selection for atrial fibrillation analysis, alarm level setting for atrial fibrillation, display area setting for atrial fibrillation burden, monitoring period setting for atrial fibrillation, and update cycle setting for atrial fibrillation burden, display threshold setting for atrial fibrillation (which means that this threshold is displayed when the atrial fibrillation burden exceeds this threshold, and this threshold is not displayed in the electrocardiogram waveform or parameter area when the atrial fibrillation burden does not exceed this threshold), etc.

The process for atrial fibrillation comprehensive management through each unit of the processor 20 is shown in FIG. 5, including the following steps.

In step 1, the atrial fibrillation analysis unit 220 acquires monitoring data of at least two vital sign parameters of a patient through the signal acquisition device 10, according to a parameter configuration of the atrial fibrillation configuration unit 210. The acquired monitoring data of the vital sign parameter includes monitoring data of electrocardiogram parameter(s). There are various ways to acquire monitoring data of the vital sign parameter, such as to acquire real-time monitoring data in method 1, and acquiring monitoring data during the preset atrial fibrillation monitoring period in method 2, which are explained below.

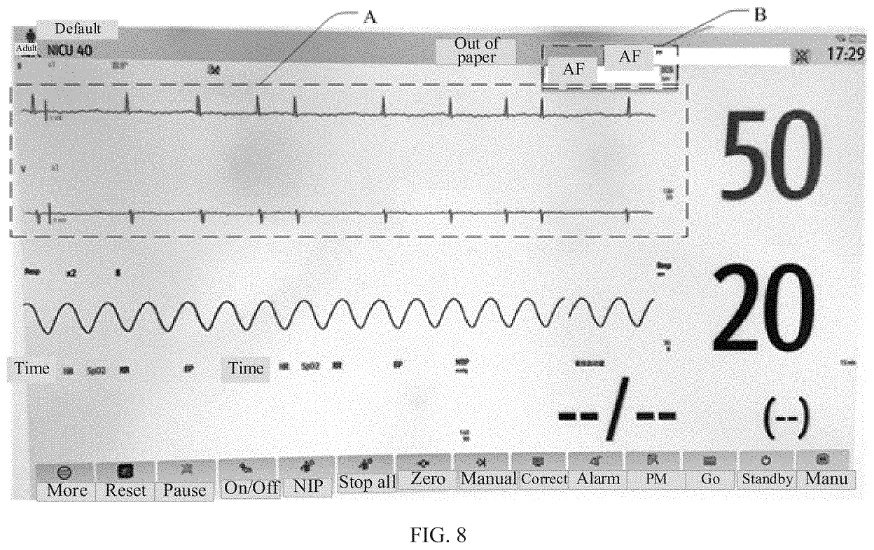

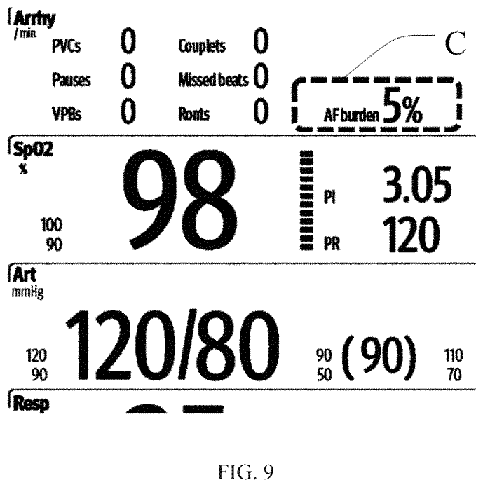

In method 1, the atrial fibrillation analysis unit 220 acquires real-time monitoring data of at least two vital sign parameters of the patient through the signal acquisition device 10. Take a sensor as an example of the signal acquisition device 10 for explanation. The sensor acquires and preprocesses the monitoring data of the vital sign parameter of the patient, including electrocardiogram parameters of one lead, to obtain corresponding real-time monitoring data. Take a communication device or interface as an example of the signal acquisition device 10 for explanation. The atrial fibrillation analysis unit 220 acquires the real-time monitoring data of the vital sign parameter from the monitor through the communication device or interface. By continuously acquiring the real-time monitoring data, the trend graph of the atrial fibrillation event can ultimately present various data within the preset atrial fibrillation monitoring period. The atrial fibrillation analysis unit 220 is further configured to display real-time electrocardiogram waveforms through the display device 30 according to the real-time monitoring data of electrocardiogram parameters, as shown in FIG. 8. The real-time electrocardiogram waveform (as shown in area A) is displayed on the real-time interface monitoring graph, facilitating daily monitoring of electrocardiogram signals of the patient by medical staff.

In method 2, the atrial fibrillation analysis unit 220 acquires the monitoring data of at least two vital sign parameters of the patient during the preset atrial fibrillation monitoring period through the signal acquisition device 10. Similarly, take a sensor as an example of the signal acquisition device 10 for explanation. The sensor acquires and preprocesses the monitoring data of the vital sign parameter of the patient during the preset atrial fibrillation monitoring period. Take a communication device or interface as an example of the signal acquisition device 10 for explanation. The atrial fibrillation analysis unit 220 acquires monitoring data of the vital sign parameter during the preset atrial fibrillation monitoring period, from the monitor through the communication device or interface. By obtaining monitoring data for various vital sign parameters during the atrial fibrillation monitoring period at one time, the trend graph of atrial fibrillation event can ultimately present various data during the preset atrial fibrillation monitoring period. The preset atrial fibrillation monitoring period can be automatically preset by the system. For example, the preset atrial fibrillation monitoring period can be a pre-stored atrial fibrillation monitoring period in the atrial fibrillation configuration unit 210, or can be inputted or modified by the user. For example, the atrial fibrillation configuration unit 210 provides a setting interface through the display device, as shown in FIG. 6. The user inputs or modifies the atrial fibrillation monitoring period through the input device in the interface shown in FIG. 6, and the atrial fibrillation configuration unit 210 acquires a new atrial fibrillation monitoring period. Then the atrial fibrillation analysis unit uses the new atrial fibrillation monitoring period to update the atrial fibrillation comprehensive view. The atrial fibrillation monitoring period can be measured in hours, days, etc. In this embodiment, the atrial fibrillation monitoring period is explained by taking 24 hours as an example.

Of course, the first and second methods are only for example, and there may be other methods. This disclosure does not limit the acquisition of monitoring data of vital sign parameter, as long as the data required for the atrial fibrillation comprehensive view can be provided.

In step 2, information of an atrial fibrillation event which occurs in the patient is acquired. There can be multiple methods, for example, if step 1 acquires the real-time monitoring data, the atrial fibrillation analysis unit 220 detects atrial fibrillation according to the acquired real-time monitoring data to obtain an atrial fibrillation analysis result, which at least includes the information of the atrial fibrillation event which occurs in the patient. Specifically, the atrial fibrillation analysis result can be obtained by the atrial fibrillation detection according to the monitoring data of electrocardiogram parameters or according to the monitoring data of other parameters, such as blood oxygen parameters, invasive blood pressure, and/or non-invasive blood pressure. The previous is explained in this embodiment. The atrial fibrillation analysis unit 220 acquires the real-time monitoring data of the electrocardiogram parameters of the patient through the signal acquisition device 10. The atrial fibrillation analysis unit 220 detects the atrial fibrillation according to the real-time monitoring data of the electrocardiogram parameters. If atrial fibrillation occurs, it generates information of the atrial fibrillation event and time duration of the atrial fibrillation. For example, the atrial fibrillation analysis unit 220 performs noise processing on the real-time monitoring data of electrocardiogram parameters; performs a P-wave analysis to determine existence, inexistence, starting and ending points, peak points, etc., of P-waves; performs F fibrillation wave analysis to determine existence and inexistence of the f-fibrillation wave; performs an irregularity analysis of RR interval; and performs heart beat classification to obtain heart beat classification result which includes: sinus, atrial, ventricular, and other categories. Specifically, a length of the RR interval is calculated based on adjacent R-waves, and the reciprocal of the length is the heart rate, then an irregularity analysis is performed on a sequence composed of RR intervals within a predetermined time period to determine whether the current paragraph of heart rate is absolutely uneven. Through one or more of the above analyses, information of atrial fibrillation event which occurs in the patient can be obtained, and the information of the atrial fibrillation event, such as the start and stop time points of the atrial fibrillation event, can be stored. The atrial fibrillation analysis unit 220 outputs the information of the atrial fibrillation event to the atrial fibrillation alarm unit 230 and the atrial fibrillation burden display unit 240. One atrial fibrillation event is one atrial fibrillation, which also corresponds to one atrial fibrillation alarm.

The monitoring system can further include buzzers, speakers, and/or warning lights. When the atrial fibrillation alarm unit 230 receives the information of the atrial fibrillation event, it indicates the atrial fibrillation occurs. The buzzers and speakers can sound an alarm indication, or the warning lights can give an alarm indication through a warning light. The display device can also display atrial fibrillation alarm information, such as displaying graphic text (an alarm string displayed in real-time in the alarm area at the top of the display interface). The alarm indication for the start and end of the atrial fibrillation can be sent to the user through the above alarm methods. As shown in FIG. 8, “atrial fibrillation” displayed in area B is a type of atrial fibrillation alarm information. The atrial fibrillation alarm can be effectively carried out by displaying the atrial fibrillation alarm information on the real-time interface monitoring graph.

The atrial fibrillation burden display unit 240 receives the information of the atrial fibrillation event outputted by the atrial fibrillation analysis unit 220, calculates the time duration of the atrial fibrillation according to the start and stop time points of the atrial fibrillation, and then calculates a real time atrial fibrillation burden according to the time duration of the atrial fibrillation and a preset update cycle. There are many methods to define the atrial fibrillation burden, such as the longest time duration of the atrial fibrillation which occurs within a preset update cycle (a preset time period), occurrence count of the atrial fibrillation within a preset update cycle (a preset time period), or percentage which is occupied by the time duration of the atrial fibrillation within a preset update cycle (a preset time period). In this embodiment, the percentage which is occupied by the time duration of the atrial fibrillation within the preset update cycle is used as the AF Burden. The preset update cycle can be automatically preset by the system, or inputted or modified by the user, and can be measured in minutes, hours, etc. In this embodiment, the update cycle is explained as 1 hour for example. The atrial fibrillation burden display unit 240 displays real-time atrial fibrillation burden on the display interface of the display device, and it can display real-time atrial fibrillation burden in the atrial fibrillation comprehensive view, as shown in the parameter trend area or typical waveform area of FIG. 2. That is, the atrial fibrillation comprehensive view also includes real-time atrial fibrillation burden information. The real-time atrial fibrillation burden can also be displayed on other display interfaces, such as being shown in the interface shown in FIG. 9. The real-time atrial fibrillation burden (AF Burden) displayed in the area C of FIG. 9 is 5%. The so-called “real-time” in real-time atrial fibrillation burden refers to refreshing according to a certain number of seconds, minutes, or hours, that is, refreshing according to the update cycle. Users can configure the update cycle, and of course, the numerical refreshing can also be performed according to the occurrence time of the atrial fibrillation alarm event.

For example, if step 1 acquires the monitoring data during the preset atrial fibrillation monitoring period, the atrial fibrillation analysis unit 220 detects the atrial fibrillation according to the acquired monitoring data to acquire the atrial fibrillation analysis result. In this embodiment, the atrial fibrillation analysis unit 220 detects the atrial fibrillation according to the monitoring data of the electrocortical parameters during the preset atrial fibrillation monitoring period to obtain the atrial fibrillation analysis result. The atrial fibrillation analysis result should at least include information of the atrial fibrillation event that occurs in the patient during the preset atrial fibrillation monitoring period. The specific process is the same as the real-time detection of atrial fibrillation mentioned above, and is not elaborated here.

In some embodiments, the atrial fibrillation analysis unit 220 can also directly acquire the real-time information of the atrial fibrillation event from the monitor through the signal acquisition device 10, or directly acquire the information of the atrial fibrillation event that occurs in the patient during the preset atrial fibrillation monitoring period from the monitor through the signal acquisition device 10.

In step 3, the atrial fibrillation analysis unit 220 displays an atrial fibrillation comprehensive view according to the acquired information of the atrial fibrillation event and the monitoring data of the at least two vital sign parameters. The atrial fibrillation comprehensive view can be automatically displayed or triggered to display under certain conditions. For example, the above content mentioned that when detecting the atrial fibrillation, the atrial fibrillation alarm unit 230 displays the atrial fibrillation alarm information through the display device 30. When the input device 40 receives a click operation of the user on the atrial fibrillation alarm information, the atrial fibrillation analysis unit 220 displays the atrial fibrillation comprehensive view through the display device 30. In other words, when the input device 40 receives the click operation of the user on the atrial fibrillation alarm information, the method proceeds to step 3. It can be seen that after seeing the alarm information of “atrial fibrillation” on the interface shown in FIG. 8, the doctor can operate the input device 40 and click on area B to present the atrial fibrillation comprehensive view, which can provide more detailed data and improve the management efficiency of doctor for the patient with atrial fibrillation.

For example, the above content mentions that the atrial fibrillation burden display unit 240 calculates the real-time atrial fibrillation burden according to the preset update cycle, and displays the real-time atrial fibrillation burden information through the display device. When the input device 40 receives the click operation of the user on the real-time atrial fibrillation burden information, the atrial fibrillation analysis unit 220 displays the atrial fibrillation comprehensive view through the display device 30. In other words, when the input device 40 receives the click operation of the user on real-time atrial fibrillation burden information, the method proceeds to step 3. It can be seen that after the doctor sees “AF burden 5%” on the interface shown in FIG. 9, he/she can operate the input device 40 and click on area C to present the atrial fibrillation comprehensive view, which provides more detailed data and improves the management efficiency of doctor for the patient with atrial fibrillation.

For example, the above content mentioned that the atrial fibrillation analysis unit 220 displays the real-time electrocardiogram waveforms through a display device according to the monitoring data of electrocardiogram parameters. When the input device 40 receives the click operation of the user on the real-time electrocardiogram waveforms, the atrial fibrillation analysis unit 220 displays the atrial fibrillation comprehensive view through the display device 30. In other words, when the input device 40 receives the click operation of the user on the real-time electrocardiogram waveforms, the method proceeds to step 3. It can be seen that after seeing the real-time electrocardiogram waveforms on the interface shown in FIG. 8, the doctor can operate the input device 40 and click on area A to present the atrial fibrillation comprehensive view, which provides more detailed data and improves the management efficiency of doctor for the patient with atrial fibrillation.

Of course, the monitoring system can also preset a hotkey. When the input device 40 receives the click operation of the user on the preset hotkey, the atrial fibrillation analysis unit 220 displays the atrial fibrillation comprehensive view through the display device, the method proceeds to step 3. It can be seen that doctors can see the atrial fibrillation comprehensive view with just one click, which is very convenient.

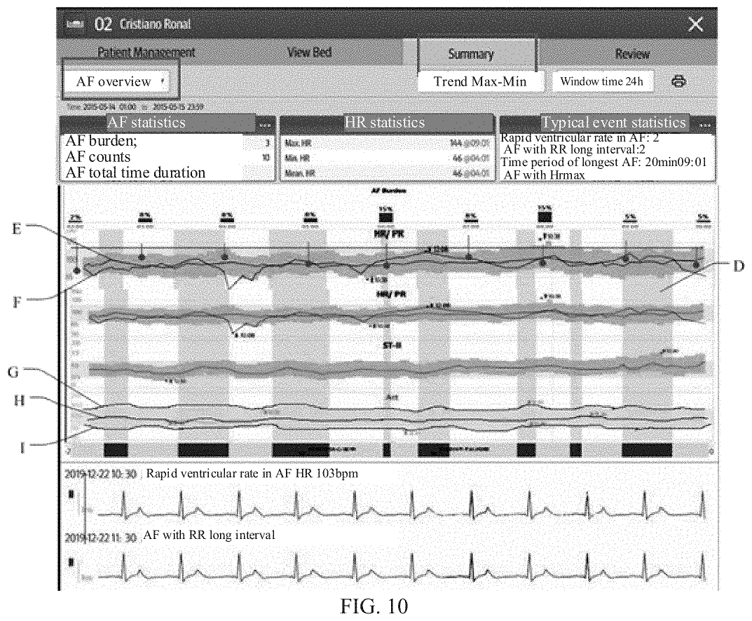

When the monitoring system monitors multiple patients, the commonly used display interface is shown in FIG. 7, where 01-05 represents number of patient support. When the input device 40 receives the patient of patient support 02, who is selected by the user, that is, when the user operates the input device by clicking on any point in the area K, the display interface redirects to the interface shown in FIG. 10. The monitoring system also provides a review function for electrocardiogram parameters. When the input device 40 receives an instruction from the user to review the overview report of the first preset time period, the atrial fibrillation analysis unit 220 acquires the monitoring data obtained by detecting one or more vital sign parameters in the first preset time period, generates an overview report according to the monitoring data of the one or more vital sign parameters in the first preset time period, and displays the overview report on the display device. Take the ECG overview as example of the overview report for explanation. The atrial fibrillation analysis unit 220 acquires monitoring data obtained by detecting electrocardiogram parameters during the first preset time period, generates the overview report according to the monitoring data of electrocardiogram parameters during the first preset time period, and displays the overview report on the display device. Doctors can review the monitoring data of electrocardiogram parameters during the first preset time period to facilitate understanding of the patient condition. Of course, the overview report can also be an overview report of other vital sign parameters, which is not elaborated here. The first preset time period can be automatically preset by the system, or can be inputted or modified by the user. As shown in FIG. 10, the user can click on the “Summary” button on the interface shown in FIG. 10 through the input device to present an overview report. The overview report includes a target virtual button, as a pull-down menu of “Atrial Fibrillation Overview” shown in FIG. 10. When the input device 40 receives the click operation of the user on the target virtual button, such as to select the “Atrial Fibrillation Overview” through the pull-down menu shown in FIG. 10, the atrial fibrillation analysis unit 220 displays the atrial fibrillation comprehensive view on the display device (the part below the pull-down menu of “Atrial Fibrillation Overview” in FIG. 10), that is, the method proceeds to step 3. At this time, the atrial fibrillation comprehensive view is displayed, and its atrial fibrillation monitoring period can be the first preset time period or a time period within the first preset time period. When reviewing electrocardiogram signals of the patient, doctor can view the atrial fibrillation comprehensive view with just one click operation to further master the atrial fibrillation situation of the patient, without the need to switch back and forth the display interface, making the operation convenient and fast.

In this embodiment, doctors can use various methods mentioned above to display the atrial fibrillation comprehensive view in the monitoring system, facilitating doctors to quickly master the patient condition.

The atrial fibrillation comprehensive view includes trend graphs 810 of at least two vital sign parameters, a trend graph 820 of the atrial fibrillation event, and an atrial fibrillation burden graph 830. The vital sign parameters displayed in the atrial fibrillation comprehensive view can be preset by the system or the user, such as displaying vital sign parameters, such as heart rate, pulse rate, blood oxygen, non-invasive blood pressure, invasive blood pressure, respiration, body temperature, ST segment information, etc.

In the atrial fibrillation comprehensive view, the trend graph 810 of at least one vital sign parameter and trend graph 820 of the atrial fibrillation event are displayed simultaneously. In this embodiment, how many vital sign parameters are obtained, the trend graphs 810 of as many vital sign parameters as obtained, are corresponding displayed, facilitating the joint presentation of the effect of the occurrence or non-occurrence of the atrial fibrillation event, on at least one vital sign parameter, at any one or more time points or within any one or more time periods during the preset atrial fibrillation monitoring period: as well as facilitating the joint presentation of the effect of the distribution of the atrial fibrillation event, on at least one vital sign parameter, at any one or more time points or within any one or more time periods during the preset atrial fibrillation monitoring period.

By comparing the trends of changes in vital sign parameters and atrial fibrillation events, it can be determined that whether the atrial fibrillation has an effect on the vital sign parameters. For example, from FIG. 2, it can be seen that as the atrial fibrillation occurs, heart rate (HR) becomes higher and higher, and then as atrial fibrillation decreases, heart rate gradually decreases, indicating that atrial fibrillation has an effect on heart rate. On the contrary, if there is no occurrence of atrial fibrillation when the heart rate increases, it indicates that the increase in heart rate is not affected by the atrial fibrillation. It can be seen that the trend graph 820 of the atrial fibrillation event and the trend graph 810 of heart rate are jointly presented, allowing doctors to more finely examine the occurrence of atrial fibrillation and whether there is a relationship between the atrial fibrillation and the changes of heart rate. Similarly, the joint presentation of the trend graph 820 of the atrial fibrillation event and the trend graph of the ST segments of the electrocardiogram waveforms can provide a more refined view of the effect of atrial fibrillation on ST. The symptoms of atrial fibrillation in organic heart disease are generally severe. When the ventricular rate exceeds 150 beats per minute, it can induce myocardial ischemia and cause angina in patient with coronary heart disease, and can lead to acute heart failure in patient with original heart dysfunction. Therefore, doctors can quickly determine whether the patient is at risk through the atrial fibrillation comprehensive view and take timely intervention measures. It can be seen that the trend graphs 810 of vital sign parameters and the trend graph 820 of the atrial fibrillation event are displayed simultaneously, facilitating doctors to quickly analyze the correlation between the two.

The trend graph 820 of the atrial fibrillation event and the atrial fibrillation burden graph 830 are displayed simultaneously, so as to jointly present a distribution difference of the atrial fibrillation event within different time periods, which have a designated atrial fibrillation burden value, during the preset atrial fibrillation monitoring period. The designated atrial fibrillation burden value can be of interest to doctor. Comparing the trend graph 820 of the atrial fibrillation event with the atrial fibrillation burden graph 830, different information can be presented. Under the same atrial fibrillation burden, the distribution of atrial fibrillation event at different time periods may vary greatly. For example, when the atrial fibrillation burden are all 50%, by combining the trend graph 820 of the atrial fibrillation event, it can be seen that the distribution and occurrence rule of atrial fibrillation events can be completely different. For example, when the atrial fibrillation burden are both 50%, when observing the trend graph 820 of the atrial fibrillation event, it may be observed that the atrial fibrillation occurs in the first half hour in some time periods, the atrial fibrillation occurs in the last half hour in some time periods, while the atrial fibrillation occurs uniformly in some time periods, etc. The comprehensive presentation of trend graph 820 of the atrial fibrillation event and atrial fibrillation burden graph 830 has clinical significance for medical staff, and is also helpful for doctor to distinguish whether the atrial fibrillation type of patient is paroxysmal, persistent, or permanent.

The trend graph 810 of at least one vital sign parameter and the atrial fibrillation burden graph 830 are displayed simultaneously, which also facilitates the joint presentation of the effect of the magnitude and/or the trend of change of the atrial fibrillation burden over time, on the at least one vital sign parameter, within any one or more time periods during the preset atrial fibrillation monitoring period. By comparing the trends of changes in the vital sign parameters and atrial fibrillation burden, it can be determined whether the atrial fibrillation burden has an effect on the vital sign parameter. For example, the atrial fibrillation burden graph 830 and the trend graph 810 of heart rate are jointly presented. Doctors can observe the effect of atrial fibrillation burden on ventricular rate and whether high heart rate is related to the atrial fibrillation burden. If the correlation is average, doctors can directly prescribe drugs to control ventricular rate. If the correlation is highly and the patient further has a relatively high ventricular rate and obvious symptoms, doctors may adjust the type of medication. In addition to simply controlling ventricular rate, they may also directly perform medication cardioversion, such as using amiodarone drugs to convert atrial fibrillation into sinus rhythm, thereby completely controlling ventricular rate. In addition, doctors may not only prescribe medication, but also make decisions to undergo atrial fibrillation ablation surgery to address the source problem. It can be seen that the joint presentation of the two is beneficial for doctors to make accurate determinations about the patient condition and take further measures.

The trend graphs 810 of at least one vital sign parameter, the trend graph 820 of the atrial fibrillation event and the atrial fibrillation burden graph 830 are displayed simultaneously, so as to jointly present a comprehensive effect of the occurrence and non-occurrence of the atrial fibrillation event and/or the distribution of the atrial fibrillation event, the magnitude and/or the trend of change of the atrial fibrillation burden over time, on the at least one vital sign parameter, during the preset atrial fibrillation monitoring period. For example, ART is arterial blood pressure, that is, a kind of IBP (invasive blood pressure) and a kind of hemodynamic parameter. Through the atrial fibrillation burden graph 830, the trend graph 820 of the atrial fibrillation event, and the trend graph of the arterial blood pressure (ART), doctors can see how much the atrial fibrillation burden or whether atrial fibrillation occurs, and their effect on hemodynamics of the circulatory system.

In this embodiment, the trend graphs 810 of vital sign parameters, the trend graph 820 of the atrial fibrillation event, and the atrial fibrillation burden graph 830 are aligned in time, as shown in FIG. 2. The three are displayed side by side and share a common time period (atrial fibrillation monitoring period). Of course, it is also possible to display one time coordinate, which is shared by the trend graphs 810 of vital sign parameters, trend graph 820 of the atrial fibrillation event, and atrial fibrillation burden graph 830. In this way, it is convenient for doctors to intuitively compare the correlation between the three.

The trend graph 820 of the atrial fibrillation event in FIG. 2 and FIG. 3 represents a single occurrence of atrial fibrillation event by a vertical line. Of course, FIG. 2 and FIG. 3 are only for illustrative purposes, and this disclosure is not limited to this. Scatter points, broken lines, curves, columns, or at least a combination of both, can be used as elements of the trend graph 820 of the atrial fibrillation event. For example, each occurrence of atrial fibrillation can be marked in the form of a point in the trend graph, and optionally, adjacent two points can be connected by a straight line for more intuitive observation. Specifically, when multiple consecutive atrial fibrillation events are marked in the form of points, these points may be connected into straight lines or curves. By observing the points representing each occurrence of atrial fibrillation event as a whole, the trend of the occurrence of the atrial fibrillation event within a predetermined time period can be known. For example, in the embodiment of FIG. 2, vertical lines with fixed length or height and perpendicular to the timeline are used as elements of the trend graph 820 of the atrial fibrillation event, representing each atrial fibrillation event, with each vertical line parallel to each other. The width of the vertical line along the timeline is configured to indicate the time duration of a single atrial fibrillation event. Therefore, if there is one or more consecutive atrial fibrillation events, the original vertical line may appear as a color block moving along the timeline. The larger the width of the color block along the timeline, the longer the time duration of the atrial fibrillation event. As shown in FIG. 3, in the trend graph 820 of the atrial fibrillation event, the relatively isolated vertical lines in the area 822 indicate that the single atrial fibrillation event has a short occurrence time and there is no regularity between various atrial fibrillation events, the relatively continuous vertical lines in area 821 indicate that the single atrial fibrillation events each have a short occurrence time and a relatively close interval, the color block in area 823 indicates that each single atrial fibrillation event has a longer duration, or the interval between multiple atrial fibrillation events is too short to be displayed in the figure. It can be seen that the trend graph 820 of the atrial fibrillation event not only presents the overall trend and state of atrial fibrillation events during the entire monitoring period, but also presents the frequency and time duration of atrial fibrillation events within different time periods. According to this, doctors can quickly determine the type of atrial fibrillation in patient. The types of atrial fibrillation usually include first-visit atrial fibrillation, paroxysmal atrial fibrillation, persistent atrial fibrillation, long-term persistent atrial fibrillation, permanent atrial fibrillation, etc.

Similarly, the trend graphs 810 of vital sign parameters can also use various elements mentioned above to present the trends of vital sign parameters, and is not further elaborated here. By using various trend graphs, such as trend graphs 810 of vital sign parameters and trend graph 820 of the atrial fibrillation event, it is easy to observe the development trend of variables to be observed (such as atrial fibrillation events, vital sign parameters, etc.) over a predetermined time period.

Specifically, the atrial fibrillation analysis unit 220 marks the occurrence time point of the atrial fibrillation event on the trend graph 820 of the atrial fibrillation event through a preset first marker to distinguish between the occurrence time point of the atrial fibrillation event and the time point when the atrial fibrillation event does not occur. For example, in the embodiment shown in FIG. 2, with the horizontal axis as the timeline, each time an atrial fibrillation event occurs, the corresponding time point on the horizontal axis is marked with a straight line of fixed length, while the time point when no atrial fibrillation event occurs is not marked. Therefore, when observing the trend graph 820 of the atrial fibrillation event over a time period as a whole, the density and distribution of the lines can be used for understanding, to a certain extent, the occurrence of atrial fibrillation events during that time period, such as the continuous occurrence, intermittent occurrence of the atrial fibrillation, etc., which is very straightforward.

When the atrial fibrillation analysis unit 220 displays through the display device 30, the trend graph 810 of at least one vital sign parameter (in this embodiment, all vital sign parameters of which the monitoring data is acquired), and the trend graph 820 of the atrial fibrillation event, a preset marker is configured to associate the atrial fibrillation event in the trend graph 820 of the atrial fibrillation event, with the vital sign parameter in the trend graph 810 of at least one vital sign parameter. The preset marker can be in various ways, such as various shapes, patterns, colors, etc., as long as it can highlight the association between the atrial fibrillation event and vital sign parameter. The preset marker shown in FIG. 2 and FIG. 3 is dashed line, while the preset marker shown in FIG. 10 is color block D. From FIG. 2 and FIG. 10, it can be seen that when doctors view the trend graph horizontally, they can see the trends of atrial fibrillation events and vital sign parameters. Without repeatedly comparing the trend graph 810 of at least one vital sign parameter, and the trend graph 820 of the atrial fibrillation event, they can also view the correlation between the atrial fibrillation event and the vital sign parameter through the preset marker.

In some embodiments, the atrial fibrillation analysis unit 220 marks a part of the trend graph 810 of at least one vital sign parameter (in this embodiment, all vital sign parameters of which the monitoring data are acquired), which part is at a time point or in a time period, when the atrial fibrillation event occurs. Therefore, when observing the trends of the vital sign parameters, doctors can determine the correlation between the vital sign parameters and the atrial fibrillation events without looking at the trend graph of atrial fibrillation event, which is very convenient. There are various ways to mark, such as using various graphics, patterns, colors, etc., as long as it can highlight the time point or time period when the atrial fibrillation event occurs.

The atrial fibrillation analysis unit 220 also processes the monitoring data of at least two vital sign parameters to identify the extremums of the at least two vital sign parameters and/or their occurrence time points, during the preset atrial fibrillation monitoring period. In this embodiment, when identifying the extremums of various vital sign parameters, the occurrence time points of the extremums are also identified correspondingly. The extremum can be either the maximum value or the minimum value. In this embodiment, the extremum includes the maximum value and the minimum value.

When the atrial fibrillation analysis unit 220 displays the trend graphs 810 of at least two vital sign parameters through the display device, the display device also presents the extremums and/or their occurrence time points of the at least two vital sign parameters. The extremums and/or their occurrence time points can be presented in various ways, such as displaying only the numerical values of the extremums and/or their occurrence time points, or only marking the positions (time points) where the extremums occur on the trend graph. Of course, it is also possible to both display the numerical values of the extremums and/or their occurrence time points, and mark the positions where the extremums occur. In this embodiment, the trend graph 810 of each vital sign parameter shows the maximum value and its occurrence time point, the minimum value and its occurrence time point. There are various ways to present the extremum, such as using various graphics, text, patterns, colors, etc. to mark it. If a time coordinate is set in the atrial fibrillation comprehensive view, marking the extremum is equivalent to marking its occurrence time point. In this embodiment, the extremum accompanied by the atrial fibrillation and the extremum without the atrial fibrillation are displayed differently. For example, the atrial fibrillation analysis unit 220 highlights through a display device the extremum and/or its occurrence time point accompanied by the atrial fibrillation, such as highlighting by color, brightness, etc. For example, the atrial fibrillation analysis unit 220 displays through the display device, the extremum and/or its occurrence time point accompanied by the atrial fibrillation with a preset first marker or text, and the extremum and/or its occurrence time point without the atrial fibrillation with a preset second marker or text. The first and second markers can be various shapes, patterns, etc., as if they can be different. Of course, the better one is that the first marker is more eye-catching than the second marker. The first marker in FIG. 2 is a triangular arrow with a fill color, and the second marker is a triangular arrow without a fill color. In this way, doctors can not only quickly find the extremums, but also quickly find which extremum is accompanied by the atrial fibrillation, which is very convenient.

The trend graphs 810 of vital sign parameters can be one of a curve graph, a histogram, a bar graph, a box line graph, a scatter graph, a broken line graph, or any combination of the curve graph, histogram, bar graph, box line graph, scatter graph, broken line graph. As shown in FIG. 2, this embodiment is illustrated using a curve graph as an example.

In some embodiments, as shown in FIG. 10, the vital sign parameters include heart rate HR and pulse rate PR. The trend graphs of heart rate and pulse rate also share the vertical axis representing the numerical value of the vital sign parameters. In FIG. 10, the heart rate curve E and pulse rate curve F share the horizontal axis and vertical axis. Patients with atrial fibrillation often have the characteristics of heart rate and pulse rate out of sync. When the heart rate curve and pulse rate curve are displayed in a superimposed manner, the superposition degree of the two curves to a certain extent reveals the severity of the symptoms of patients with atrial fibrillation, which facilitate doctors to find the progress of patient with atrial fibrillation as soon as possible.

In some embodiments, the vital sign parameters include at least two of: systolic blood pressure, diastolic blood pressure, and mean blood pressure, and their trend graphs also share a vertical coordinate representing the numerical value of the vital sign parameters. In FIG. 10, the vital sign parameters include systolic blood pressure, diastolic blood pressure, and mean blood pressure. The trend graphs of the three are G, 1, and H, which share the horizontal axis (time) and vertical axis (numerical magnitude).

The atrial fibrillation analysis unit 220 can also distinguish and display on the display device, the trend graphs of vital sign parameters sharing a common vertical axis by using different colors. For example, the heart rate curve E and pulse rate curve F in FIG. 2 are distinguished by different colors. The atrial fibrillation analysis unit 220 can also distinguish the trend graphs of vital sign parameters displayed through the display device on a common vertical axis using different line types (such as thick solid lines, thin solid lines, various dashed lines, dotted lines, etc.). For example, in FIG. 10, the systolic blood pressure curve G and diastolic blood pressure curve I are displayed by solid lines, while the mean blood pressure curve H is displayed by dashed lines, for distinguishing. This makes it easier to distinguish curves that share a common ordinate.

The atrial fibrillation comprehensive view further includes at least one of: statistical information for atrial fibrillation, statistical information for vital sign parameter, statistical information for typical event, and typical waveform information, as shown in FIG. 2. This embodiment includes the above four types. The atrial fibrillation analysis unit performs statistics on atrial fibrillation related information during the preset atrial fibrillation monitoring period to obtain statistical information for atrial fibrillation. The statistical information for atrial fibrillation can be presented in the form of text, graphs, etc. In this embodiment, it is presented in the form of atrial fibrillation statistical charts, as shown in the information statistics area of FIG. 2. Among them, the statistical information for atrial fibrillation includes at least one of the following: total occurrence count of the atrial fibrillation during the preset atrial fibrillation monitoring period, total time duration of the atrial fibrillation during the preset atrial fibrillation monitoring period, total atrial fibrillation burden during the preset atrial fibrillation monitoring period, occurrence count of the atrial fibrillation during at least one preset time period of the preset atrial fibrillation monitoring period, time duration of the atrial fibrillation during at least one preset time period of the preset atrial fibrillation monitoring period, atrial fibrillation burden during at least one preset time period of the preset atrial fibrillation monitoring period, statistical information segmented for time duration of the atrial fibrillation during the preset atrial fibrillation monitoring period. Doctors can accurately and detailly understand atrial fibrillation status of the patient during the monitoring period through statistical information for atrial fibrillation, which is very convenient.

The atrial fibrillation analysis unit makes statistics on the monitoring data of vital sign parameter during the preset atrial fibrillation monitoring period, and acquires statistics information for the vital sign parameter. The statistics information for the vital sign parameter includes at least one of the following: extremum of the at least one vital sign parameter and its occurrence time point, average value of the at least one vital sign parameter, and statistical information segmented, of the at least one vital sign parameter. Similarly, the statistics information for the vital sign parameter can be presented in the form of text, graph, etc. FIG. 2 shows the statistical chart for the heart rate in the preset atrial fibrillation monitoring period.

The atrial fibrillation analysis unit performs statistics on typical events during the preset atrial fibrillation monitoring period, and acquires statistical information for typical event. Statistical information for typical event include at least one of the following: count of rapid ventricular rate in the atrial fibrillation, count of atrial fibrillation with RR long interval(s), count of extreme tachycardia, count of extreme bradycardia, a time point of first-onset atrial fibrillation, a time point of atrial fibrillation with maximum heart rate, and an occurrence time period of the longest atrial fibrillation. Typical events are usually events that doctors focus on and have clinical significance. The atrial fibrillation comprehensive view can also include an alarm event statistical chart during the preset atrial fibrillation monitoring period. The alarm event statistical chart can reflect various alarms generated by the monitoring system during patient monitoring. Of course, it also includes atrial fibrillation alarm events. Alarm events are also typical events, and the alarm event statistical chart can be displayed as a part of the statistical information for typical event in the information statistics area of FIG. 2.

The atrial fibrillation analysis unit processes the monitoring data of electrocardiogram signals to obtain typical waveform information. Typical waveform information includes at least one fragment of at least one of the following waveforms: electrocardiogram waveform(s) of first-onset atrial fibrillation of the patient, electrocardiogram waveform(s) containing a highest heart rate during the preset atrial fibrillation monitoring period, electrocardiogram waveform(s) containing a lowest heart rate during the preset atrial fibrillation monitoring period, and electrocardiogram waveform(s) corresponding to atrial fibrillation of longest time duration during the preset atrial fibrillation monitoring period. Typical waveform information can be presented to doctors as typical electrocardiogram waveforms, and has clinical significance.

The specific types of the statistical information for atrial fibrillation, the statistical information for vital sign parameter, the statistical information for typical waveform, and the statistical information for typical event, can be set in the atrial fibrillation configuration unit, or according to the system default settings.

The atrial fibrillation analysis unit 220 is further configured to determine a target time period selected by operation of the user on the atrial fibrillation comprehensive view, and display, through the display, the electrocardiogram waveforms within the target time period. For example, the trend graphs of various vital sign parameters can be selected horizontally (in the time). The user can move the cursor to the extremum position through the input device, or click on the extremum position to display the electrocardiogram waveform(s) for a time period before and after the extremum. The electrocardiogram waveforms within the target time period can be displayed as typical waveform information in the typical waveform area, and of course, it can also be displayed in other areas. It can be seen that operation of the user based on the trend graphs can be displayed in conjunction with typical waveform information, and a large amount of information can be displayed on a limited display interface.

In some embodiments, the trend graph of vital sign parameter includes a trend graph for heart rate, and the preset atrial fibrillation monitoring period includes multiple time periods of minimum unit, as shown in FIG. 11. The time period corresponding to each bar in the atrial fibrillation burden graph 830 is a time period of minimum unit. The atrial fibrillation analysis unit 220 responds to selection operation for a target time segment to further display an atrial fibrillation view segmented for time 840 through the display device. Users can choose target time segments in various forms, such as directly inputting the target time segment through the input device, or selecting the target time segment on the trend graph 810 of vital sign parameter, trend graph 820 of the atrial fibrillation event, or atrial fibrillation burden graph 830 through the input device. The target time segment can be any of the multiple time periods of minimum unit, as shown in FIG. 11 and FIG. 12. The user can operate the input device to click on the atrial fibrillation burden graph 830, and the corresponding time period of minimum unit is the target time segment. Of course, the target time segment can also include multiple consecutive time periods of minimum unit. The user can operate the input device to slide and select a distance on the atrial fibrillation burden graph 830, and the multiple time periods of minimum unit corresponding to this distance are the target time segment. It can be seen that users can select the time period they are interested in when viewing the atrial fibrillation comprehensive view, thereby presenting the atrial fibrillation time-segmented view 840, and the information display is very fast. As shown in FIG. 11, the atrial fibrillation time-segmented view 840 can be displayed around the atrial fibrillation comprehensive view, or as a floating window on the atrial fibrillation comprehensive view. Of course, it can also be displayed on a separate display interface.

The atrial fibrillation time-segmented view 840 includes at least one of: trend graph for heart rate 841 during the target time segment, statistical information for heart rate 842 during the target time segment, and statistical information for typical heart rate event during the target time segment. It can be seen that doctors can see detailed information about their heart rate during a period of interest, which is beneficial for doctors to assess the patient condition. The statistical information for heart rate 842 can be presented in the form of attached figures, as shown in FIGS. 1 and 12, and of course, it can also be presented in text or list form. The atrial fibrillation time-segmented view 840 also includes at least one of: statistical information for atrial fibrillation during the target time segment and the statistical information for typical atrial fibrillation event during the target time segment. It can be seen that the atrial fibrillation time-segmented view 840 is equivalent to a local view of the atrial fibrillation comprehensive view. Doctors can see the comprehensive information of heart rate and atrial fibrillation within the target time segment, making it easy to accurately assess the patient condition.

The trend graph of vital sign parameter can also include a target value marker, which is configured to identify the target value of vital sign parameter. The target value can be preset by the system or set and modified by the user. Taking the trend graph for heart rate as an example, as shown in FIG. 2, the target value marker of the trend graph for heart rate is the target heart rate marker, which is configured to mark the preset target heart rate. The target heart rate can be preset by the system or set and modified by the user. The target heart rate can be the maximum value or minimum value of the heart rate, and can also include the maximum value and minimum value of the heart rate. Correspondingly, the target heart rate marker can be the maximum value maker or minimum value marker or include both the maximum value maker and minimum value marker. The target heart rate marker can be text, symbols, graphics, and patterns. The target heart rate marker in FIG. 2 is the corresponding marking line for the target heart rate. Doctors can intuitively see whether the heart rate of the patient meets the standard according to the target heart rate marker, which is very convenient.

Correspondingly, the trend graph for heart rate 841 not only includes the heart rate curve within the target time segment, but also includes the target heart rate marker. In FIGS. 11 and 12, there are two target heart rate markers: target line 1 and target line 2. From the figures, it can be seen at a glance that the heart rate of the patient exceeds the target heart rate during the target time segment, having a good indication effect.

The processor can also generate an overview report containing the atrial fibrillation comprehensive view and other information according to the information of the atrial fibrillation event and monitoring data of various vital sign parameters. The overview report (electronic version) can be displayed and outputted to other devices, and the printing device connected to the processor can also be controlled to print the overview report. Even in some embodiments, the overview report, atrial fibrillation comprehensive view, etc., may not be displayed, but can be provided to doctors by outputting an electronic version of the overview report or printing a paper overview report through a printing device.

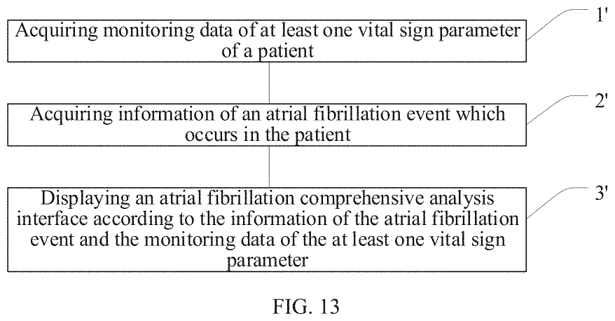

In the above embodiment, the signal acquisition device of the monitoring system acquires monitoring data of at least two vital sign parameters. In this embodiment, the signal acquisition device can also only acquire monitoring data of electrocardiogram parameter. Specifically, this disclosure also provides a monitoring data display method, as shown in FIG. 13, including the following steps.

In step 1′, the atrial fibrillation analysis unit 220 acquires monitoring data of at least one vital sign parameter of a patient through the signal acquisition device 10, according to a parameter configuration of the atrial fibrillation configuration unit 210. The at least one vital sign parameter includes electrocardiogram parameters, that is, in the embodiment shown in FIG. 13, just monitoring data of electrocardiogram parameter can be obtained, or monitoring data of multiple vital sign parameters including the electrocardiogram parameter can also be obtained. In addition, this step is the same as step 1 of the above embodiment and is not elaborated here.

In step 2′, information of the atrial fibrillation event that occurs in the patient, is acquired. This step is the same as Step 2 of the above embodiment and is not elaborated here.

In step 3′, the atrial fibrillation analysis unit 220 displays an atrial fibrillation comprehensive analysis interface according to the acquired information of the atrial fibrillation event and the monitoring data of at least one vital sign parameter, as shown in FIG. 2. The atrial fibrillation comprehensive analysis interface includes a parameter trend area and/or information statistics area.

Wherein, the parameter trend area displays the trend graph 810 of at least one vital sign parameter, and the parameter trend area also displays at least one of the trend graph 820 of the atrial fibrillation event and the atrial fibrillation burden graph 830. This embodiment takes the parameter trend area as an example to display the trend graph 820 of the atrial fibrillation event and the atrial fibrillation burden graph 830. The trend graph of the at least one vital sign parameter, the trend graph of the atrial fibrillation event, and the atrial fibrillation burden graph are displayed simultaneously, so as to jointly present a correlation between at least two of the at least one vital sign parameter, the atrial fibrillation event that occurs in the patient, and the atrial fibrillation burden, at any one or more time points or within any one or more time periods, during the preset atrial fibrillation monitoring period. The specific content displayed in the parameter trend area is elaborated in detail in the above embodiments, and is not repeated here.

The information statistics area displays at least one of followings: statistical information for atrial fibrillation, statistical information for the at least one vital sign parameter, and statistical information for typical event. The specific content displayed in the information statistics area is elaborated in detail in the above embodiments, and is not repeated here.

The atrial fibrillation comprehensive analysis interface also includes a typical waveform area, which displays typical waveform information. The specific content of typical waveform information is elaborated in detail in the above embodiments, and is not repeated here.

As shown in FIG. 2, the atrial fibrillation comprehensive analysis interface mainly presents the parameter trend area, while the information statistics area and/or typical waveform area can be set around the parameter trend area. Of course, the information statistics area and/or typical waveform area can also be hidden, and the information statistics area and/or typical waveform area are presented when triggered by operation of the user (such as moving the cursor out of the parameter trend area).

In summary, this disclosure provides a monitoring system, atrial fibrillation comprehensive management method and monitoring data display method etc. Through the design of the atrial fibrillation comprehensive view (AF View), information, such as atrial fibrillation event alarms, atrial fibrillation burden, and vital sign parameters, can be jointly presented, which not only qualitatively and quantitatively presents information related to atrial fibrillation, but also reflects the correlation between the atrial fibrillation and vital sign parameters. At the same time, the centralized presentation of statistical data, such as the distribution of atrial fibrillation events during the patient monitoring cycle etc., provides assistance for medical staff to systematically evaluate the physiological and pathological status of patients, facilitating them to more systematically and finely evaluate the changes in atrial fibrillation status of patient over a time period, improving the efficiency of displaying atrial fibrillation related data for patient, and also improving the intervention efficiency of doctor in patients with atrial fibrillation.

This disclosure refers to various exemplary embodiments for explanation. However, those skilled in this field can recognize that changes and modifications can be made to exemplary embodiments without departing from the scope of this disclosure. For example, various operational steps and components configured to execute them can be implemented in different ways according to specific applications or considering any number of cost functions associated with system operations (for example, one or more steps can be deleted, modified, or combined into other steps).

In addition, as understood by those skilled in the art, the principles herein can be reflected in a computer program product on a computer-readable storage medium which is pre-loaded with computer-readable program code. Any tangible, non-transitory computer-readable storage medium can be used, including magnetic storage devices (hard disk, floppy disk, etc.), optical storage devices (CD-ROM, DVD, Blu ray disk, etc.), flash memory and/or the likes. Computer program instructions can be loaded onto a general-purpose computer, a special-purpose computer, or other programmable data processing device to form a machine, so that these instructions executed on a computer or other programmable data processing device can generate a device to realize a specified function. These computer program instructions may also be stored in a computer-readable memory, which may instruct a computer or other programmable data processing device to operate in a specific manner, so that the instructions stored in the computer-readable memory can form a manufactured article, including an implementation device for realizing a specified function. Computer program instructions can also be loaded on a computer or other programmable data processing device, so that a series of operation steps are performed on the computer or other programmable device to generate a computer implemented process, so that the instructions executed on the computer or other programmable device can provide steps for realizing the specified functions.

Although the principles herein have been shown in various embodiments, many modifications of structures, arrangements, proportions, elements, materials, and components that are particularly applicable to specific environmental and operational requirements may be used without departing from the principles and scope of the present disclosure. The above modifications and other changes or amendments are included in the scope of this disclosure.

The foregoing specific description has been described with reference to various embodiments. However, those skilled in the art can recognize that various amendments and changes may be made without departing from the scope of this disclosure. Accordingly, consideration of this disclosure is illustrative rather than restrictive, and all such modifications are included within its scope. Similarly, there are solutions to the advantages, other advantages, and problems of the various embodiments as described above. However, the benefits, advantages, solutions to problems and any solution that can produce these elements or make them more explicit should not be interpreted as critical, necessary or necessary. The term “include” and any other variations thereof as used herein are non-exclusive inclusions, so that a process, method, article or device that includes a list of elements includes not only these elements, but also other elements that are not explicitly listed or do not belong to the process, method, system, article or device. In addition, the term “connection” and any other variation thereof as used herein refer to physical connection, electrical connection, magnetic connection, optical connection, communication connection, functional connection and/or any other connection.

Those skilled in the art recognize that many changes can be made to the details of the above embodiments without departing from the basic principles of this disclosure. Therefore, the scope of this disclosure shall be determined according to the following attached claims.

1. An atrial fibrillation comprehensive management method, comprising:
acquiring monitoring data of at least two vital sign parameters of a patient, wherein the at least two vital sign parameters comprise one or more electrocardiogram parameters;
acquiring information of an atrial fibrillation event occurring in the patient;
displaying an atrial fibrillation comprehensive view, according to the information of the atrial fibrillation event and the monitoring data of the at least two vital sign parameters; wherein the atrial fibrillation comprehensive view comprises trend graphs of the at least two vital sign parameters, a trend graph of the atrial fibrillation event, and an atrial fibrillation burden graph;
wherein the trend graphs of the at least two vital sign parameters are configured to present changes of the at least two vital sign parameters during a preset atrial fibrillation monitoring period, the trend graph of the atrial fibrillation event is configured to present the atrial fibrillation event that occurs in the patient during the preset atrial fibrillation monitoring period, and the atrial fibrillation burden graph is configured to present atrial fibrillation burden during at least one time period of minimum unit within the preset atrial fibrillation monitoring period;
wherein the method further comprises:
simultaneously displaying the trend graphs of the at least two vital sign parameters, the trend graph of the atrial fibrillation event, and the atrial fibrillation burden graph, so as to jointly present a correlation between at least two of: at least one vital sign parameter, the atrial fibrillation event that occurs in the patient, and the atrial fibrillation burden, at any one or more time points or within any one or more time periods during the preset atrial fibrillation monitoring period.

2. The method according to claim 1, wherein simultaneously displaying the trend graphs of the at least two vital sign parameters, the trend graph of the atrial fibrillation event, and the atrial fibrillation burden graph, so as to jointly present a correlation between at least two of: at least one vital sign parameter, the atrial fibrillation event that occurs in the patient, and the atrial fibrillation burden, at any one or more time points or within any one or more time periods during the preset atrial fibrillation monitoring period, comprises:
displaying the trend graph of the at least one vital sign parameter and the trend graph of the atrial fibrillation event simultaneously, so as to jointly present an effect of occurrence and non-occurrence of the atrial fibrillation event and distribution of the atrial fibrillation event, on the at least one vital sign parameter, at any one or more time points or within any one or more time periods during the preset atrial fibrillation monitoring period;
displaying the trend graph of the atrial fibrillation event and the atrial fibrillation burden graph simultaneously, so as to jointly present a distribution difference of the atrial fibrillation event within different time periods with a designated atrial fibrillation burden value during the preset atrial fibrillation monitoring period;
displaying the trend graph of the at least one vital sign parameter and the atrial fibrillation burden graph simultaneously, so as to jointly present an effect of a magnitude of the atrial fibrillation burden or of a trend of change of the atrial fibrillation burden over time, on the at least one vital sign parameter, within any one or more time periods during the preset atrial fibrillation monitoring period; or
simultaneously displaying the trend graph of the at least one vital sign parameter, the trend graph of the atrial fibrillation event, and the atrial fibrillation burden graph, so as to jointly present a comprehensive effect of occurrence and non-occurrence of the atrial fibrillation event or of distribution of the atrial fibrillation event, of a magnitude of the atrial fibrillation burden or of a trend of change of the atrial fibrillation burden over time, on the at least one vital sign parameter, during the preset atrial fibrillation monitoring period.

3. The method according to claim 1, wherein the trend graph of the atrial fibrillation event marks an occurrence time point of the atrial fibrillation event through a preset first marker to distinguish between the occurrence time point of the atrial fibrillation event and a time point when the atrial fibrillation event does not occur.

4. The method according to claim 1, further comprising:
marking a part of the trend graph of the at least one vital sign parameter at a time point or within a time period when the atrial fibrillation event occurs; or
associating, by using a preset marker, the atrial fibrillation event in the trend graph of the atrial fibrillation event with the at least one vital sign parameter in the trend graph of the at least one vital sign parameter, when displaying the trend graph of the at least one vital sign parameter and the trend graph of the atrial fibrillation event.

5. The method according to claim 1, further comprising:
processing the monitoring data of the at least two vital sign parameters to identify extremums of the at least two vital sign parameters or their occurrence time points during the preset atrial fibrillation monitoring period; and
further presenting the extremums of the at least two vital sign parameters or their occurrence time points, when displaying the trend graphs of the at least two vital sign parameters.

6. The method according to claim 5, further comprising:
displaying the extremums or their occurrence time points which are accompanied by the atrial fibrillation event, in a highlighted way or by using a preset first marker or text; or
displaying the extremums or their occurrence time points which are not accompanied by the atrial fibrillation event, by using a preset second marker or text.

7. (canceled)

8. The method according to claim 1, wherein;
the trend graph of the vital sign parameter comprises: a curve graph, a histogram, a bar graph, a box graph, a scatter graph, a broken line graph, or a combination thereof; or
the trend graph of the vital sign parameter comprises a trend graph of a heart rate: the atrial fibrillation comprehensive view further comprises a target heart rate marker configured to mark a preset target heart rate; or
the trend graph of the vital sign parameter comprises at least one of: a trend graph of heart rate, a trend graph of pulse rate, a trend graph of blood oxygen, a trend graph of non-invasive blood pressure, a trend graph of invasive blood pressure, a trend graph of respiration, a trend graph of body temperature, a trend graph of cardiac output per stroke, a trend graph of cardiac output, a trend graph of ST segment of electrocardiogram, a trend graph of QT interval of electrocardiogram, a trend graph of blood sugar, a trend graph of cerebral oxygen, or a trend graph of urine output: or
the atrial fibrillation comprehensive view further comprises at least one of: an atrial fibrillation statistical chart during the preset atrial fibrillation monitoring period, a heart rate statistical chart during the preset atrial fibrillation monitoring period, or an alarm event statistical chart during the preset atrial fibrillation monitoring period.

9. The method according to claim 1, wherein the at least two vital sign parameters comprise a heart rate and a pulse rate, and a trend graph of the heart rate and a trend graph of the pulse rate share a vertical coordinate representing a numerical magnitude of the at least two vital sign parameters: or
the at least two vital sign parameters comprise at least two of; systolic blood pressure, diastolic blood pressure, or mean blood pressure, and their trend graphs share a vertical coordinate representing a numerical magnitude of the at least two vital sign parameters.

10. The method according to claim 9, further comprising:
displaying, in different colors or line patterns, the trend graphs of the at least two vital sign parameters which share a vertical coordinate.

11. The method according to claim 1, wherein the atrial fibrillation comprehensive view further comprises at least one of; statistical information for the atrial fibrillation, statistical information for the vital sign parameter, statistical information for a typical event, or typical waveform information.

12. The method according to claim 11, wherein the statistical information for the atrial fibrillation comprises at least one of: total occurrence count of the atrial fibrillation during the preset atrial fibrillation monitoring period, total time duration of the atrial fibrillation during the preset atrial fibrillation monitoring period, total atrial fibrillation burden during the preset atrial fibrillation monitoring period, occurrence count of the atrial fibrillation during at least one preset time period within the preset atrial fibrillation monitoring period, time duration of the atrial fibrillation during at least one preset time period within the preset atrial fibrillation monitoring period, atrial fibrillation burden during at least one preset time period within the preset atrial fibrillation monitoring period, statistical information segmented for time duration of the atrial fibrillation during the preset atrial fibrillation monitoring period; or
the statistical information for the vital sign parameter comprises an extremum or its occurrence time point, an average value, and segmented statistical information, of the at least one vital sign parameter; or
the statistical information for the typical event comprises at least one of: count of rapid ventricular rate in the atrial fibrillation, count of atrial fibrillation with RR long interval, count of extreme tachycardia, count of extreme bradycardia, a time point of first-onset atrial fibrillation, a time point of atrial fibrillation with maximum heart rate, and an occurrence time period of longest atrial fibrillation; or
the typical waveform information comprises at least one fragment of at least one of following: an electrocardiogram waveform of first-onset atrial fibrillation of the patient, an electrocardiogram waveform containing a highest heart rate during the preset atrial fibrillation monitoring period, an electrocardiogram waveform containing a lowest heart rate during the preset atrial fibrillation monitoring period, or an electrocardiogram waveform corresponding to atrial fibrillation of longest time duration during the preset atrial fibrillation monitoring period.

13-14. (canceled)

15. The method according to claim 1, further comprising:
determining a target time period which is selected by operation of a user on the atrial fibrillation comprehensive view, and displaying electrocardiogram waveform(s) within the target time period;
receiving a new atrial fibrillation monitoring period which is inputted by a user, and updating the atrial fibrillation comprehensive view using the new atrial fibrillation monitoring period; or
acquiring, in real time, the monitoring data of the one or more electrocardiogram parameters of the patient: detecting the atrial fibrillation according to the monitoring data of the one or more electrocardiogram parameters to obtain time duration of the atrial fibrillation, and calculating real-time atrial fibrillation burden information, according to the time duration of the atrial fibrillation and a preset update cycle, wherein the atrial fibrillation comprehensive view further comprises the real-time atrial fibrillation burden information.

16-17. (canceled)

18. The method according to claim 1, wherein the preset atrial fibrillation monitoring period comprises multiple time periods of minimum unit;
the trend graph of the vital sign parameter comprises a trend graph of a heart rate;
the method further comprises:
responding to operation of a user for selecting a target time segment to further display an atrial fibrillation view segmented for time,
wherein the atrial fibrillation time-segmented view comprises: at least one of the trend graph of the heart rate during the target time segment, statistical information for the heart rate during the target time segment, and statistical information for typical heart rate event during the target time segment; and at least one of statistical information for atrial fibrillation during the target time segment or statistical information for atrial fibrillation typical event during the target time segment,
wherein the target time segment is any one of the multiple time periods of minimum unit, or comprises multiple continuous time periods of minimum unit.

19. (canceled)

20. The method according to claim 1, wherein the acquired monitoring data of the one or more electrocardiogram parameters are real-time monitoring data: wherein acquiring information of the atrial fibrillation event occurring in the patient comprises detecting the atrial fibrillation according to the real-time monitoring data of the one or more electrocardiogram parameters, so as to acquire the information of the atrial fibrillation event which occurs in the patient;
wherein the method further comprises:
displaying atrial fibrillation alarm information when detecting the atrial fibrillation, and displaying the atrial fibrillation comprehensive view, when receiving click operation of a user on the atrial fibrillation alarm information; or
calculating real-time atrial fibrillation burden according to a preset update cycle and displaying real-time atrial fibrillation burden information, and displaying the atrial fibrillation comprehensive view, when receiving click operation of a user on the real-time atrial fibrillation burden information; or
displaying real-time an electrocardiogram waveform according to the real-time monitoring data of the one or more electrocardiogram parameters, and displaying the atrial fibrillation comprehensive view, when receiving click operation of a user on the real-time electrocardiogram waveform.

21. The method according to claim 1, further comprising:
displaying the atrial fibrillation comprehensive view; when receiving click operation of a user on a preset hotkey; or
when receiving an instruction from a user to review an overview report of a first preset time period, acquiring the monitoring data of the one or more electrocardiogram parameters during the first preset time period, generating the overview report according to the monitoring data of the one or more electrocardiogram parameters during the first preset time period, and displaying the overview report,
wherein the overview report comprises a target virtual button, and the method further comprises: displaying the atrial fibrillation comprehensive view, when receiving click operation of the user on the target virtual button.

22-24. (canceled)

25. A monitoring data display method, comprising:
displaying an atrial fibrillation comprehensive analysis interface, according to information of an atrial fibrillation event and monitoring data of at least one vital sign parameter, wherein the atrial fibrillation comprehensive analysis interface comprises a parameter trend area or an information statistics area,
wherein the parameter trend area displays a trend graph of the at least one vital sign parameter, and the parameter trend area further displays at least one of: a trend graph of the atrial fibrillation event or an atrial fibrillation burden graph; wherein the trend graph of the at least one vital sign parameter is configured to present a change of the at least one vital sign parameter during a preset atrial fibrillation monitoring period, the trend graph of the atrial fibrillation event is configured to present the atrial fibrillation event that occurs in the patient during the preset atrial fibrillation monitoring period, and the atrial fibrillation burden graph is configured to present atrial fibrillation burden during at least one time period of minimum unit within the preset atrial fibrillation monitoring period;
wherein the method further comprises:
displaying the trend graph of the at least one vital sign parameter simultaneously with at least one of the trend graph of the atrial fibrillation event or the atrial fibrillation burden graph, so as to jointly present a correlation between the at least one vital sign parameter, and at least one of the atrial fibrillation event that occurs in the patient and the atrial fibrillation burden, at any one or more time points or within any one or more time periods during the preset atrial fibrillation monitoring period;
wherein the information statistics area displays at least one of statistical information for the atrial fibrillation, statistical information for the at least one vital sign parameter, or statistical information for a typical event.

26. The method according to claim 25, wherein displaying the trend graph of the at least one vital sign parameter simultaneously with at least one of the trend graph of the atrial fibrillation event or the atrial fibrillation burden graph, so as to jointly present a correlation between the at least one vital sign parameter, and at least one of the atrial fibrillation event that occurs in the patient and the atrial fibrillation burden, at any one or more time points or within any one or more time periods during the preset atrial fibrillation monitoring period, comprises:
displaying the trend graph of the at least one vital sign parameter and the trend graph of the atrial fibrillation event simultaneously, so as to jointly present an effect of occurrence and non-occurrence of the atrial fibrillation event and distribution of the atrial fibrillation event, on the at least one vital sign parameter, at any one or more time points or within any one or more time periods during the preset atrial fibrillation monitoring period;
displaying the trend graph of the atrial fibrillation event and the atrial fibrillation burden graph simultaneously, so as to jointly present a distribution difference of the atrial fibrillation event within different time periods with a designated atrial fibrillation burden value during the preset atrial fibrillation monitoring period;
displaying the trend graph of the at least one vital sign parameter and the atrial fibrillation burden graph simultaneously, so as to jointly present an effect of a magnitude of the atrial fibrillation burden or of a trend of change of the atrial fibrillation burden over time, on the at least one vital sign parameter, within any one or more time periods during the preset atrial fibrillation monitoring period; or
simultaneously displaying the trend graph of the at least one vital sign parameter, the trend graph of the atrial fibrillation event and the atrial fibrillation burden graph, so as to jointly present a comprehensive effect of occurrence and non-occurrence of the atrial fibrillation event or of distribution of the atrial fibrillation event, of a magnitude of the atrial fibrillation burden or of a trend of change of the atrial fibrillation burden over time, on the at least one vital sign parameter, during the preset atrial fibrillation monitoring period.

27. (canceled)

28. The method according to claim 25, further comprising:
marking a part of the trend graph of the at least one vital sign parameter at a time point or within a time period when the atrial fibrillation event occurs: or associating, by using a preset marker, the atrial fibrillation event in the trend graph of the atrial fibrillation event with the at least one vital sign parameter in the trend graph of the at least one vital sign parameter, when displaying the trend graph of the at least one vital sign parameter and the trend graph of the atrial fibrillation event; or
processing the monitoring data of the at least one vital sign parameter to identify extremums of the at least one vital sign parameter or their occurrence time points during the preset atrial fibrillation monitoring period; and further presenting the extremums of the at least one vital sign parameter or their occurrence time points, when displaying the trend graph of the at least one vital sign parameter; or
displaying the atrial fibrillation comprehensive analysis interface, when receiving click operation of a user on a preset hotkey; or when receiving an instruction from a user to review an overview report of a first preset time period, acquiring the monitoring data of one or more electrocardiogram parameters during the first preset time period, generating the overview report according to the monitoring data of the one or more electrocardiogram parameters during the first preset time period, and displaying the overview report, wherein the overview report comprises a target virtual button, and
the method further comprises: displaying the atrial fibrillation comprehensive analysis interface, when receiving click operation of the user on the target virtual button.

29-30. (canceled)

31. The method according to claim 28, wherein the acquired monitoring data of the one or more electrocardiogram parameters are real-time monitoring data;
wherein the method further comprises:
acquiring information of an atrial fibrillation event occurring in the patient, including detecting the atrial fibrillation according to the real-time monitoring data of the one or more electrocardiogram parameters, so as to acquire the information of the atrial fibrillation event which occurs in the patient; and
wherein the method further comprises:
displaying atrial fibrillation alarm information when detecting the atrial fibrillation, and displaying the atrial fibrillation comprehensive analysis interface, when receiving click operation of a user on the atrial fibrillation alarm information; or
calculating real-time atrial fibrillation burden according to a preset update cycle and displaying real-time atrial fibrillation burden information, and displaying the atrial fibrillation comprehensive analysis interface, when receiving click operation of a user on the real-time atrial fibrillation burden information; or
displaying a real-time electrocardiogram waveform according to the monitoring data of the one or more electrocardiogram parameters, and displaying the atrial fibrillation comprehensive analysis interface, when receiving click operation of a user on the real-time electrocardiogram waveform.

32. (canceled)

33. An atrial fibrillation comprehensive management method, comprising:
acquiring monitoring data of at least one vital sign parameter of a patient, wherein the at least one vital sign parameter comprises one or more electrocardiogram parameters;
acquiring information of an atrial fibrillation event occurring in the patient; and
outputting an electronic version or a paper version of an overview report according to the information of the atrial fibrillation event and the monitoring data of the at least one vital sign parameter, wherein the overview report comprises a trend graph of the at least one vital sign parameter, as well as a trend graph of the atrial fibrillation event or an atrial fibrillation burden graph,
wherein the trend graph of the at least one vital sign parameter is configured to present a change of the at least one vital sign parameter during a preset atrial fibrillation monitoring period, the trend graph of the atrial fibrillation event is configured to present the atrial fibrillation event that occurs in the patient during the preset atrial fibrillation monitoring period, and the atrial fibrillation burden graph is configured to present atrial fibrillation burden during at least one time period of minimum unit within the preset atrial fibrillation monitoring period.

34-62. (canceled)
